(19) United States
(12) Patent Application Publication
(10) Pub. No.: US 2023/0190403 A1
(43) Pub. Date: Jun. 22, 2023

(54) SYSTEM FOR MONITORING A SURGICAL LUMINAIRE ASSEMBLY

(71) Applicant: KARL LEIBINGER MEDIZINTECHNIK GMBH & CO. KG, Muehlheim (DE)

(72) Inventor: Joachim STROELIN, Rietheim-Weilheim (DE)

(21) Appl. No.: 18/000,095

(22) PCT Filed: May 27, 2021
(86) PCT No.: PCT/EP2021/064204

§ 371 Date: Nov. 28, 2022

(30) Foreign Application Priority Data
May 29, 2020 (DE) 10 2020 114 418.0

(51) Int. Cl. A61B 90/30; F21V 23/04

(52) U.S. Cl. CPC A61B 90/30; F21V 23/0492; F21W 2131/205

(57) ABSTRACT

The present invention comprises a system for monitoring a surgical luminaire assembly comprising at least one surgical luminaire, by means of a monitoring unit. It is provided here that the system comprises a sensor array, wherein the monitoring unit determines the position of the at least one surgical luminaire and/or the orientation of a light axis of the at least one surgical luminaire by evaluating the data of the sensor array.

The present invention relates to the operation of a surgical luminaire assembly comprising at least one surgical luminaire or at least two surgical luminaires.

In an operating theatre, in most cases, in addition to the surgical luminaire assembly, which is usually mounted on the ceiling via a support system, many other devices are provided, some of which are also mounted on the ceiling, and some of which are located in the area below the surgical luminaire assembly. In addition, there are usually several operating theatre staff.

Collisions occur repeatedly and regularly when positioning the surgical luminaires or other devices. On the one hand, the collisions prevent the desired positioning and, on the other hand, the collisions can cause damage to the devices. This can be quite critical if, over a longer period of time, the damage causes paint to flake off and fall off. In the case of massive collisions, parts can come loose or even arms can break.

Surgical luminaires of new design continue to have a very high performance potential. The maximum illuminance as well as the maximum irradiance are capped by the surgical luminaire standard 60601-2-41. The standard stipulates a maximum irradiance of 1000 W/m² per surgical luminaire unit. However, this limit is not sufficient to reliably prevent heating of the surgical field and the associated accelerated tissue drying.

The operating theatre is not usually equipped with sensors. At most, there is a normal room camera or cameras in the surgical luminaires to document an operating theatre. Furthermore, there are navigation systems to position the instruments and their direction during difficult or complex operations.

DE 10 2007 028 731 A1 presents a method in which three-dimensional image data of an operating theatre are captured and objects/devices are separated in order to determine motion sequences from the available and assignable data sets and to intervene in operating functions in a controlling manner.

Document DE 10 2014 212 632 A1 presents a method for monitoring the operation of a medical device. In particular, the configuration of an operating table is ascertained via a 3D sensor and transferred to a kinematic model of the operating table.

It is an object of the present invention to provide a system that allows safer and/or simpler operation of the surgical luminaire assembly.

This object is achieved by a system for monitoring a surgical luminaire assembly according to claim 1. Preferred embodiments of the present invention are the subject-matter of the dependent claims.

The present invention comprises a system for monitoring a surgical luminaire assembly comprising at least one surgical luminaire, by means of a monitoring unit. According to the invention, the system further comprises a sensor array, wherein the monitoring unit determines the position of the at least one surgical luminaire and/or the orientation of a light axis of the at least one surgical luminaire by evaluating the data of the sensor array. The detection of the position of the surgical luminaire and/or the orientation of the light axis according to the invention allows a safer and/or simpler operation of the surgical luminaire assembly in several possible application scenarios.

In one possible embodiment of the present invention, the monitoring unit determines the absolute position of the at least one surgical luminaire and/or the absolute orientation of a light axis in space by evaluating the data of the sensor array. In particular, the position of the at least one surgical luminaire and/or the orientation of a light axis in a spatial coordinate system of the operating theatre is therefore determined.

On the one hand, this allows flexible processing of the position and/or orientation for different scenarios and purposes. Furthermore, reliable sensors are available for this purpose. Furthermore, the determination can be made independently for the surgical luminaire in question.

In one possible embodiment of the present invention, the surgical luminaire assembly comprises at least two surgical luminaires, wherein the system determines the relative position and/or orientation of the light axes of the surgical luminaires in relation to each other.

Preferably, the system determines the relative position and/or orientation of the light axes of the surgical luminaires in relation to each other from the absolute positions of the surgical luminaires and/or the absolute orientations of the light axes in space, determined for each of the surgical luminaires.

In one possible embodiment of the present invention, the system determines whether and/or at what distance the light axes of the surgical luminaires intersect and/or the light fields of the surgical luminaires overlap.

In one possible embodiment of the present invention, it is provided that the system performs an acoustic or optical indication and/or controls an illuminance of the surgical luminaires depending on whether the light axes intersect and/or the light fields of the surgical luminaires overlap.

In one possible embodiment of the present invention, it is provided that when the light axes intersect and/or the light fields of the surgical luminaires overlap, the system emits an acoustic or optical warning and/or automatically limits or reduces the illuminance of the lamps depending on a total energy input of the lamps, a total illuminance of the lamps and/or a parameter derived therefrom.

In one possible embodiment of the present invention, it is provided that the monitoring unit determines the energy input into the surgical field depending on the light field diameter and the illuminance of each of the at least two surgical luminaires.

In one possible embodiment of the present invention, it is provided that the sensor array detects the position of the at least one surgical luminaire, in particular the absolute position in space.

For example, the sensor array can have one or more of the following embodiments:

In one possible embodiment of the present invention, the at least one surgical luminaire comprises navigation points, the position of which is detected by a sensor in order to determine the orientation and position of the surgical luminaire.

In one possible embodiment of the present invention, the at least one surgical luminaire comprises a sensor which detects the orientation of the surgical luminaire, in particular a sensor for determining the absolute orientation of the surgical luminaire, in particular the orientation relative to the earth's magnetic field and/or the force of gravity.

In one possible embodiment of the present invention, the at least one surgical luminaire is arranged on a support system, wherein the support system comprises one or more joints, the position of which is detected via a sensor in order to determine the position and/or orientation of the surgical luminaire, wherein the sensor is in particular an angle sensor and/or rotary encoder.

In one possible embodiment of the present invention, the sensor array comprises a 3D sensor which detects the position and/or orientation of the at least one surgical luminaire.

In particular, the 3D sensor detects the surgical luminaire and from this the position and/or orientation of the at least one surgical luminaire. The 3D sensor can be arranged here in a fixed position, in particular on a central shaft of the support system or on a wall or ceiling of the operating theatre.

In one possible embodiment of the present invention, the surgical luminaire assembly comprises at least two surgical luminaires, each of which comprises an independent system for position and/or orientation detection and an interface for communication with each other.

In one possible embodiment of the present invention, the surgical luminaire assembly comprises at least two surgical luminaires, wherein a common monitoring unit is provided which evaluates the signals of the sensor array.

In one possible embodiment of the present invention, sensors and/or navigation points of the sensor array are arranged on a housing of the at least one surgical luminaire, wherein the sensors and/or navigation points are preferably retrofittable.

The navigation points are preferably optical markers. These can be configured as two-dimensional images and/or three-dimensional bodies.

In one possible embodiment of the present invention, the at least one surgical luminaire is arranged on a support system, wherein a sensor for detecting the position and/or orientation of the at least one surgical luminaire is arranged on a central bearing shaft of the support system.

In particular, this is a sensor for detecting navigation points located on the surgical luminaires and/or a 3D sensor.

In one possible embodiment of the present invention, the system monitors the at least one surgical luminaire for collisions with other surgical luminaires of the surgical luminaire assembly and/or other items of equipment.

In one possible embodiment of the present invention, a surgical field can be pre-set and the system determines at least one orientation and/or position of the at least one surgical luminaire by which the surgical field is illuminated, wherein the orientation and/or position is indicated acoustically and/or optically and/or is approached by one or more drives.

In one possible embodiment of the present invention, the surgical luminaire is arranged on a support system, wherein the support system comprises one or more driven joints by which the position and/or orientation of the surgical luminaire is approached.

In one possible embodiment of the present invention, the system monitors the position and/or orientation of the surgical luminaire assembly with respect to the function of a ventilation ceiling. Such a ventilation ceiling is typically positioned above the operating table and is intended to generate a downwardly directed laminar airflow of purified air which surrounds the surgical zone and which is intended to prevent contaminants from entering the operating area. The position and/or orientation of the surgical luminaire assembly can have a significant effect on the airflow and is therefore monitored in accordance with the invention.

In one possible embodiment of the present invention, the system emits a warning when the function of the ventilation ceiling is impaired.

In one possible embodiment of the present invention, the monitoring unit changes the position and/or orientation of the surgical luminaire assembly if the function of the ventilation ceiling is impaired.

In one possible embodiment of the present invention, the system controls the ventilation ceiling depending on the position and/or orientation of the surgical luminaire assembly, in particular to maintain the function of the ventilation ceiling despite the position and/or orientation of the luminaire assembly.

In one possible embodiment of the present invention, the monitoring unit comprises a microcontroller and software which is stored on a non-volatile memory and which runs on the microcontroller to implement the functions described above. For this purpose, the monitoring unit is connected to the sensor array to receive and evaluate signals from the sensor array. Furthermore, the monitoring unit may be connected to input and/or output elements and/or a controller of the system.

Further, in one possible embodiment of the present invention, the system comprises a controller comprising a microcontroller and software which is stored on a non-volatile memory and which runs on the microcontroller to implement the control functions described above.

The monitoring unit can be integrated into the controller or formed separately therefrom.

The present invention further comprises a surgical luminaire assembly comprising at least one surgical luminaire and a system as described above.

The present invention will now be explained in greater detail with the aid of a drawing and exemplary embodiments.

In the drawing:

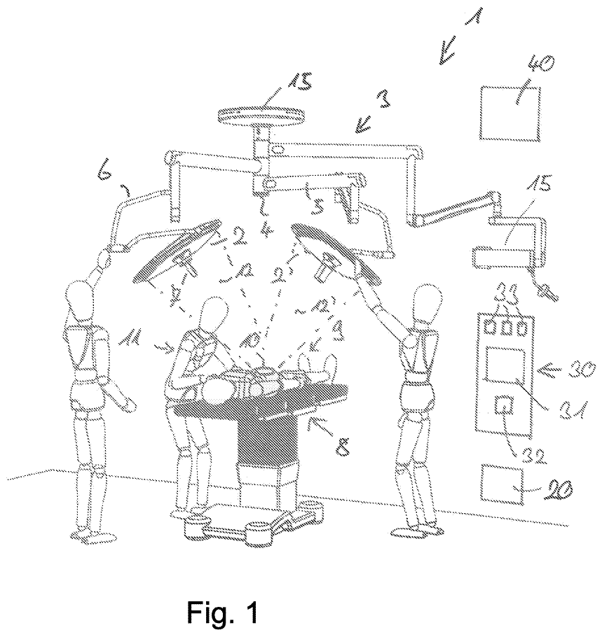

FIG. 1 shows an exemplary embodiment of a surgical luminaire assembly according to the invention with a monitoring unit according to the invention; and

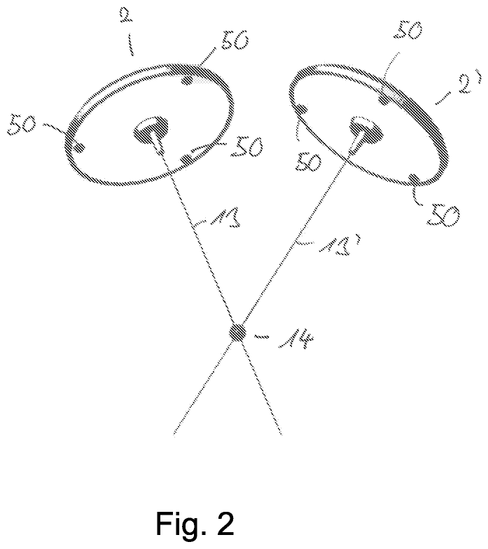

FIG. 2 shows a schematic representation of two surgical luminaires with navigation points and the orientation of the light axes.

FIG. 1 shows an exemplary embodiment of a surgical luminaire assembly 1 according to the invention comprising a first surgical luminaire 2 and a second surgical luminaire 2′. However, within the scope of the present invention, the surgical luminaire assembly 1 could also comprise only one surgical luminaire or more than two surgical luminaires.

In the exemplary embodiment, the surgical luminaires 2 and 2′ are adjustable in their position and orientation via a support system 3 above an operating table 8. The adjustment is usually done by hand. However, adjustment by means of drives of the support system 3 is also conceivable. In the exemplary embodiment, the support system comprises a ceiling mount 15, via which a central shaft 4 is mounted on the ceiling. Support arms 5 are pivotably arranged on the central shaft 4. The surgical luminaires 2 and 2′ are each arranged on different support arms 5 via further support arm elements 6 and joints, and have a handle 7 on which they can be moved. However, other designs of the support system are also conceivable.

The surgical luminaires 2 and 2′ each generate a light field 12 and 12′ respectively with a light axis 13 and 13′ respectively. By arranging and orienting the surgical luminaires 2 and 2′ accordingly, the light fields 12 and 12′ can be directed towards a surgical zone 10 of the patient 9 lying on the operating table 8 so that they overlap. In other cases, however, the two light fields 12 and 12′ of the surgical luminaires 2 and 2′ can also be directed towards different areas. For example, in the context of a transplantation, one surgical luminaire can be directed towards the surgical zone 10 of the patient 9 lying on the operating table 8, and another surgical luminaire can be directed towards the transplant.

FIG. 1 shows a control unit 30 via which functions of the surgical luminaires 2 and 2′ can be controlled, in particular brightness adjustment and/or light field size and/or color temperature and/or switching on and off. In the exemplary embodiment shown, this control unit is mounted on a wall. Alternatively, the control unit 30 could also be embodied as a table or mobile version. The control unit preferably has input elements 33, for example in the form of switches, actuators and/or a touchscreen. Furthermore, the control unit 30 preferably comprises a display 31 on which operating states and/or current setting parameters of the individual surgical luminaires 2 and 2′ can be displayed.

The surgical luminaires 2 and 2′ can be networked with each other and/or with a common controller and/or operating unit by cable and/or wirelessly. Via this communication, it is preferably possible to control and/or synchronize functions of the surgical luminaires 2 and 2′, such as brightness adjustment, focus adjustment or color temperature, as well as simultaneous switching on and off.

According to the invention, the surgical luminaire assembly comprises a monitoring unit 20, which is only shown symbolically here. This can be part of a controller for the surgical luminaires, integrated therein and/or external thereto, and/or of the control unit 11. Furthermore, a sensor array 40, which is also shown only schematically, is provided, wherein the monitoring unit 20 determines the position of the at least one surgical luminaire and/or the orientation of a light axis of the at least one surgical luminaire by evaluating the data of the sensor array 40.

In particular, the monitoring unit can be configured to determine the absolute position of the at least one surgical luminaire in space and/or the absolute orientation of a light axis in space by evaluating the data of the sensor array.

In the exemplary embodiment shown in FIG. 2, there are at least three navigation points 50 on each of the surgical luminaires 2 and 2′. Based on the three positions of the navigation points 50 in space, both the position of the surgical luminaire 2 or 2′ in space and the orientation of the light axis 13 or 13′ of the surgical luminaire can be determined. From the data of two or more surgical luminaires, the monitoring unit determines whether these light axes 13 and 13′ intersect/cross and at what distance this intersection point 14 lies. The maximum illuminance can then also be expected at this point.

If an overlap of the light fields 12 and 12′ and/or an intersection of the light axes 13 and 13′ is detected, warnings can be given (visually or acoustically) or the luminaires can be dimmed or other parameters can be changed, depending on further parameters such as the total illuminance and/or the total energy input of the surgical luminaires into the surgical zone 10. In particular, the warnings can be issued via the control unit 30.

Furthermore, it is conceivable to equip other mobile and/or adjustable devices in the operating theatre with navigation points. For example, one or more of the following devices can be equipped with navigation points: operating theatre table 8, anesthesia station, monitor suspension means 15, one or more support systems 3 of the operating theatre lights 2, 2′, supply systems (CSU=ceiling supply unit) with electrical and gas connections (1-2 pieces per operating theatre) and/or their support systems, various equipment trolleys, one or more instrument tables, stools, steps, infusion stands, endoscopy trolleys, monitor trolleys.

For example, a table or operating table 8 or a cabinet can be equipped with navigation points at the top four corners. Or, another support system of a ceiling supply unit 15 is equipped with navigation points at the ends of the booms and/or a support platform.

In one possible embodiment, these navigation points 50 can be attached externally to the surgical luminaires and/or equipment, for example magnetically, glued, or screwed. In this way, even an older operating theatre can be easily retrofitted.

These navigation points 50 are detected by at least one sensor of the sensor array 40. The detection can take place optically, acoustically (ultrasound) or via electromagnetic waves, in particular radio waves, for example. The detection of the navigation points 50 is carried out by one or more sensors. These sensors can be cameras, 3D sensors, ultrasonic sensors, radio wave receivers or magnetic field sensors.

The navigation points can be passive elements or active elements. Passive elements are detected by the sensors of the sensor unit without becoming active themselves. Active elements actively send signals that are detected by the sensors of the sensor unit.

In one possible embodiment, the navigation points can be optical markers that are detected by an optical sensor of the sensor array, in particular a 3D sensor. In particular, they can be two-dimensional markers or three-dimensional objects. The markers can be at least partially coded.

Furthermore, the monitoring unit has a computer for evaluating the data.

Ideally, all components of the system are networked with this computer. This computer analyses the data and calculates, for example, the best possible settings and positions.

In one possible embodiment, the smallest unit is an evaluation of two surgical luminaires 2, 2′. Both the position detection and evaluation systems are located in and/or on the surgical luminaires themselves. The luminaires communicate with each other to exchange information about their respective positions and/or orientations. The surgical luminaires determine via the communication with the other surgical luminaire whether their light axes 13, 13′ cross and/or the light fields 12, 12′ are congruent. In one possible embodiment, the detection and evaluation is integrated into the luminaire electronics.

In one possible embodiment, the sensors/navigation points are attached externally on the luminaire housing.

In one possible embodiment, the sensor for detecting the position and/or orientation of the surgical luminaires and/or for detecting the navigation points is attached to the central bearing shaft 4 on the support system 3. In one possible embodiment, the computer for evaluation is provided externally, and is integrated into the control unit 30, for example.

In FIG. 2, the navigation points 50 are arranged on an underside of the surgical luminaires 2, 2′. In this case, the sensor array is preferably arranged below the surgical luminaire assembly. In other exemplary embodiments, the navigation points 50 are arranged on an outer edge and/or on an upper side of the surgical luminaires 2, 2′. In this way, they can be detected via a sensor array which is arranged to the side of and/or above the surgical luminaires.

Alternatively or in addition to the use of navigation points 50, other technologies are also conceivable, via which the position and/or orientation of the surgical luminaires 2, 2′ is detected:
to determine the position and/or orientation of the surgical luminaires (or any other device arranged on the support system 3), all joints of the support system 3 are equipped with sensors, for example in the form of protractors/rotary encoders;
to determine the position and/or orientation of the surgical luminaires 2, 2′ (or another device), these are equipped with acceleration and/or inclination sensors and/or an electronic compass and/or an electronic spirit level, as known from smartphones.

A great many functions can be derived from the information on the position and/or orientation of the surgical luminaires 2, 2′ and/or the other devices.

The monitoring unit 20 can, for example, be configured to warn of collisions. Alternatively or additionally, the support system 3 and/or the devices can have brakes and/or drives, wherein the monitoring unit stops the drives and/or applies the brakes in order to avoid collisions.

Furthermore, the monitoring unit can calculate optimal positions with the help of a computer. The support system 3 and/or the devices can have drives that are controlled by the monitoring unit in order to assume the optimal positions.

In particular, surgical luminaires which are arranged on a support system with driven arms, booms and/or joints can assume an optimum position through the control by means of the monitoring unit and can thus be positioned between the surgeons 11 with the best possible aiming towards the surgical zone 10, so that no shadows are created.

The monitoring unit may be configured to recognize when the operator 11 repositions himself and then to reposition the surgical luminaire.

In one possible embodiment of the present invention, the monitoring unit 20 uses the information from the sensor array to determine where and at what height the operating table with patient is and at what distance therefrom the surgical luminaire(s) 2, 2′ is/are located. Preferably, the monitoring unit uses this information to determine an orientation and/or control of the surgical luminaire(s), by means of which they are optimally focused on the surgical zone 10.

In one possible embodiment of the present invention, predefined scenarios are stored in the monitoring unit and are approached depending on the sensor data, for example predefined scenarios for different operating theatre disciplines. For example, a scenario could be defined as follows: 3 persons present. These are detected, and the operating table 8 and the surgical luminaires 2, 2′ are arranged in an associated predefined position and corresponding parameters are pre-set at all devices.

In one possible embodiment of the present invention, the monitoring unit uses the information determined by the sensor unit to optimally adjust a ventilation ceiling arranged on the ceiling above the operating table, or issues notifications if the function of the ventilation ceiling is disturbed, for example, by an unfavorable positioning of the surgical luminaires 2, 2′ and/or a large obstacle.

1. A system for monitoring a surgical luminaire assembly comprising at least one surgical luminaire, by means of a monitoring unit,
wherein
the system comprises a sensor array, wherein the monitoring unit determines the position of the at least one surgical luminaire and/or the orientation of a light axis of the at least one surgical luminaire by analyzing the data of the sensor array.

2. The system according to claim 1, wherein the surgical luminaire assembly comprises at least two surgical luminaires, and wherein the system determines the relative position and/or orientation of the light axes of the surgical luminaires in relation to each other.

3. The system according to claim 2, wherein the system determines whether and/or at what distance the light axes of the surgical luminaires intersect and/or the light fields of the surgical luminaires overlap.

4. The system according to claim 3, wherein the system performs an acoustic or optical indication and/or controls an illuminance of the surgical luminaires depending on whether the light axes intersect and/or the light fields of the surgical luminaires overlap.

5. The system according to claim 4, wherein when the light axes intersect and/or the light fields of the surgical luminaires overlap, the system emits an acoustic or optical warning and/or automatically limits or reduces the illuminance of the lamps depending on a total energy input of the lamps, a total illuminance of the lamps and/or a parameter derived therefrom.

6. The system according to claim 1, wherein the sensor array detects the position of the at least one surgical luminaire.

7. The system according to claim 1, wherein the sensor array comprises at least one of the following configurations:
the at least one surgical luminaire comprises navigation points, the position of which is detected by a sensor in order to determine the orientation and position of the surgical luminaire;
the at least one surgical luminaire comprises a sensor which detects the orientation of the surgical luminaire, in particular a sensor for determining the orientation relative to the earth's magnetic field and/or the force of gravity;
the at least one surgical luminaire is arranged on a support system, wherein the support system comprises one or more joints, the position of which is detected via a sensor in order to determine the position and/or orientation of the surgical luminaire, wherein the sensor is in particular an angle sensor and/or rotary encoder;
the sensor array comprises a 3D sensor which detects the position and/or orientation of the at least one surgical luminaire.

8. The system according to claim 1, wherein the surgical luminaire assembly comprises at least two surgical luminaires, each of which comprises an independent system for position and/or orientation detection and an interface for communication with each other.

9. The system according to claim 1, wherein the surgical luminaire assembly comprises at least two surgical luminaires, wherein a common monitoring unit is provided which evaluates the signals of the sensor array.

10. The system according to claim 1, wherein sensors and/or navigation points of the sensor array are arranged on a housing of the at least one surgical luminaire.

11. The system according to claim 1, wherein the at least one surgical luminaire is arranged on a support system, wherein a sensor for detecting the position and/or orientation of the at least one surgical luminaire is arranged on a central bearing shaft of the support system, in particular a sensor for detecting navigation points located on the surgical luminaires, and/or a 3D sensor.

12. The system according to claim 1, wherein the at least one surgical luminaire is monitored for collisions with other surgical luminaires of the surgical luminaire assembly and/or other items of equipment.

13. The system according to claim 1, wherein a surgical field can be pre-set and the system determines at least one orientation and/or position of the at least one surgical luminaire by which the surgical field is illuminated, wherein the orientation and/or position is indicated acoustically and/or optically and/or is approached by one or more drives, and/or wherein the surgical luminaire is arranged on a support system, wherein the support system comprises one or more driven joints by which the position and/or orientation of the surgical luminaire is approached.

14. The system according to claim 1, wherein the system monitors the position and/or orientation of the surgical luminaire assembly with respect to the function of a ventilation ceiling.

15. A surgical luminaire assembly comprising at least one surgical luminaire and a system according to claim 1.

16. The system according to claim 5, wherein the monitoring unit determines the energy input into the surgical field depending on the light field diameter and the illuminance of each of the at least two surgical luminaires.

17. The system according to claim 14 wherein the system emits a warning if the function is impaired and/or changes the position and/or orientation of the surgical luminaire assembly if the function is impaired and/or controls the ventilation ceiling depending on the position and/or orientation of the surgical luminaire assembly.

18. The system according to claim 10, wherein the sensors and/or navigation points are retrofittable.
